(19) United States
(12) Patent Application Publication
(10) Pub. No.: US 2026/0029408 A1
(43) Pub. Date: Jan. 29, 2026

(54) METHODS FOR DETECTING ANTI-PORCINE ANTIBODIES

(71) Applicant: NEW YORK UNIVERSITY, New York, NY (US)

(72) Inventor: Massimo MANGIOLA, New York, NY (US)

(73) Assignee: NEW YORK UNIVERSITY, New York, NY (US)

(21) Appl. No.: 19/122,850

(22) PCT Filed: Oct. 23, 2023
(86) PCT No.: PCT/US2023/035702

§ 371 Date: Apr. 21, 2025

Related U.S. Application Data
(60) Provisional application No. 63/418,276, filed on Oct. 21, 2022.

(51) Int. Cl. G01N 33/68; G01N 33/543; G01N 33/567

(52) U.S. Cl. CPC G01N 33/6854; G01N 33/54346; G01N 33/567; G01N 33/6893; G01N 2800/245; G01N 2800/50; G01N 2800/52

(57) ABSTRACT

The present application provides methods of predicting the risk of a subject developing a xenograft rejection response, methods of monitoring the risk of a subject developing a xenograft rejection response, methods of identifying a new porcine antigen(s) recognized by anti-porcine antibodies, methods for creating a porcine xenograft donor with a minimized likelihood of inducing a xenograft rejection response, as well as reagents and kits suitable for use in the described methods.

### CROSS-REFERENCE TO RELATED APPLICATIONS

This application claims priority to U.S. Provisional Application Ser. No. 63/418,276 filed on Oct. 21, 2022, the contents of which is incorporated herein by reference in their entirety.

### FIELD OF THE INVENTION

The present invention relates to methods for detecting anti-porcine antibodies in patients receiving xenotransplantation.

### BACKGROUND

Xenograft from genetically modified pigs is one of the most promising solutions to the shortage of human organs available to patients awaiting transplantation¹. However, the biggest challenge to this model, is the presence of pre-formed anti-pig antibodies in human sera and the risk of hyperacute rejection¹. Virtually all humans express antibodies directed against xenoantigens. However, each person expresses a different amount and type of immunoglobulins. For example, every human expresses anti-galactose-α1,3-galactose (αGAL) antibodies, and most people express a discrete amount of both anti-SDa glycan and anti-N-glycolylneuraminic acid (Neu5Gc) antibodies. Many will express a detectable quantity of antibodies directed against xenoantigens that are still unknown. Additionally, an individual transplanted with a pig organ not only can increase the pre-existing antibodies but is also projected to develop a number or new antibodies targeting xenoantigens (neo-antigens) that are still unknown. Therefore, every individual expresses anti-pig antibodies with different clinical relevance. For xenotransplantation to move from clinical studies to clinical practice, detection of anti-pig antibodies needs to be scalable and rapid and cannot rely solely on cell-based assays. Rapid and precise identification and characterization of xeno-antibodies is essential for risk stratification of xenotransplant candidates. Longitudinal examination of post-transplant changes in xeno-sensitization is of paramount importance for clinical applications of xenotransplantation.

### SUMMARY OF THE INVENTION

In one aspect, the present disclosure provides, among other things, a method of predicting the risk of a subject developing a xenograft rejection response, wherein the subject is considered for a xenograft transplantation from a porcine donor, said method comprising:
a) contacting a sample obtained from the subject with a test solid or semisolid support comprising immobilized thereon cell membranes isolated from one or more porcine cells, wherein the porcine cells are isolated from the porcine donor or another animal within the same breeding family.
b) determining a titer of IgG and IgM antibodies in the sample obtained from the subject, wherein the titer is determined as the dilution at which the amount of IgG and IgM antibodies bound to the test solid or semisolid support is not more than that of a negative control sample, and
c) determining the risk of the subject developing the xenograft rejection response based on the titer determined in step (b).

In some embodiments of the method described above, step (c) comprises determining:
(1) the subject is at a low risk of developing the xenograft rejection response when the sample has a titer of less than or equal to 1:8;
(2) the subject is at a moderate risk of developing the xenograft rejection response when the sample has a titer of higher than 1:8 but less than 1:32 (e.g., less than or equal to 1:16, or higher than 1:16 but less than 1:32); or
(3) the subject is at a high risk of developing the xenograft rejection response when the sample has a titer of higher than or equal to 1:32.

In some embodiments of the method described above, the method further comprises proceeding with the xenograft transplantation from the porcine donor, when the subject is determined to be at a low risk of developing the xenograft rejection response.

In some embodiments of the method described above, the method further comprises performing a treatment to reduce the amount of anti-porcine IgG and IgM antibodies in the blood of the subject prior to performing the xenograft transplantation from the porcine donor, when the subject is determined to be at a moderate risk of developing the xenograft rejection response.

In some embodiments of the method described above, the method further comprises not proceeding with the xenograft transplantation from the porcine donor or performing a treatment to reduce the amount of anti-porcine IgG and IgM antibodies in the blood of the subject prior to performing the xenograft transplantation from the porcine donor, when the subject is determined to be at a high risk of developing the xenograft rejection response.

In another aspect, provided herein is a method of predicting the risk of a subject developing a xenograft rejection response, wherein the subject is considered for a xenograft transplantation from a porcine donor, said method comprising:
a) contacting a sample obtained from the subject with a test solid or semisolid support comprising immobilized thereon one or more Swine Leukocyte Antigens (SLA),
b) determining a titer of IgG and IgM antibodies in the sample obtained from the subject, wherein the titer is determined as the dilution at which the amount of IgG and IgM antibodies bound to the test solid or semisolid support is not more than that of a negative control sample, and
c) determining the risk of the subject developing the xenograft rejection response based on the titer determined in step (b).

In some embodiments of the method described above, step (c) comprises determining:
(1) the subject is at a low risk of developing the xenograft rejection response when the sample has a titer of less than or equal to 1:16;
(2) the subject is at a moderate risk of developing the xenograft rejection response when the sample has a titer of higher than or equal to 1:16 but less than 1:32; or
(3) the subject is at a high risk of developing the xenograft rejection response when the sample has a titer of higher than or equal to 1:32.

In some embodiments of the method described above, the method further comprises proceeding with the xenograft transplantation from the porcine donor, when the subject is determined to be at a low risk of developing the xenograft rejection response.

In some embodiments of the method described above, the method further comprises performing a treatment to reduce the amount of anti-porcine IgG and IgM antibodies in the blood of the subject prior to performing the xenograft transplantation from the porcine donor, when the subject is determined to be at a moderate risk of developing the xenograft rejection response.

In some embodiments of the method described above, the method further comprises not proceeding with the xenograft transplantation from the porcine donor or performing a treatment to reduce the amount of anti-porcine IgG and IgM antibodies in the blood of the subject prior to performing the xenograft transplantation from the porcine donor, when the subject is determined to be at a high risk of developing the xenograft rejection response.

In another aspect, provided herein is a method of predicting the risk of a subject developing a xenograft rejection response, wherein the subject is considered for a xenograft transplantation from a porcine donor, said method comprising:
a) contacting a sample obtained from the subject with a test solid or semisolid support comprising immobilized thereon one or more of galactose-α1,3-galactose (αGAL). N-glycolylneuraminic acid (Neu5Gc) (any known variant and subtypes), and/or the SDa blood group (SDa) (any known variant and subtypes),
b) determining a titer of IgG and IgM antibodies in the sample obtained from the subject, wherein the titer is determined as the dilution at which the amount of IgG and IgM antibodies bound to the test solid or semisolid support is not more than that of a negative control sample, and
c) determining the risk of the subject developing the xenograft rejection response based on the titer determined in step (b).

In some embodiments of the method described above, the test solid or semisolid support comprises immobilized thereon Neu5Gc (any known variant and subtypes) and/or SDa (any known variant and subtypes).

In some embodiments of the method described above, step (c) comprises determining:
(1) the subject is at a low risk of developing the xenograft rejection response when the sample has a titer of less than or equal to 1:16;
(2) the subject is at a moderate risk of developing the xenograft rejection response when the sample has a titer of higher than or equal to 1:16 but less than 1:32; or
(3) the subject is at a high risk of developing the xenograft rejection response when the sample has a titer of higher than or equal to 1:32.

In some embodiments of the method described above, the method further comprises proceeding with the xenograft transplantation from the porcine donor, when the subject is determined to be at a low or moderate risk of developing the xenograft rejection response.

In some embodiments of the method described above, the method further comprises performing a treatment to reduce the amount of anti-porcine IgG and IgM antibodies in the blood of the subject prior to performing the xenograft transplantation from the porcine donor, when the subject is determined to be at a high risk of developing the xenograft rejection response.

In another aspect, the present disclosure provides a method of predicting the risk of a subject developing a xenograft rejection response, wherein the subject is considered for a xenograft transplantation from a porcine donor, said method comprising:
a) contacting a sample obtained from the subject with i) a test solid or semisolid support having immobilized thereon cell membranes isolated from one or more porcine cells, wherein the porcine cells are isolated from the porcine donor or another animal within the same breeding family, or ii) a test solid or semisolid support having immobilized thereon one or more Swine Leukocyte Antigens (SLA), and/or iii) a test solid or semisolid support having immobilized thereon one or more of galactose-α1,3-galactose (αGAL), N-glycolylneuraminic acid (Neu5Gc), and/or the SDa blood group (SDa) (e.g., Neu5Gc and SDa).
b) determining a titer of IgG and IgM antibodies in the sample obtained from the subject, wherein the titer is determined as the dilution at which the amount of IgG and IgM antibodies bound to the test solid or semisolid support is not more than that of a negative control sample, and
c) determining the risk of the subject developing the xenograft rejection response based on the titer determined in step (b).

In some embodiments of the method described above, step (a) comprises contacting a sample obtained from the subject with i) a test solid or semisolid support having immobilized thereon cell membranes isolated from one or more porcine cells, wherein the porcine cells are isolated from the porcine donor or another animal within the same breeding family, and ii) a test solid or semisolid support having immobilized thereon one or more Swine Leukocyte Antigens (SLA).

In some embodiments of the method described above, step (a) comprises contacting a sample obtained from the subject with i) a test solid or semisolid support having immobilized thereon cell membranes isolated from one or more porcine cells, wherein the porcine cells are isolated from the porcine donor or another animal within the same breeding family, and ii) a test solid or semisolid support having immobilized thereon one or more of galactose-α1,3-galactose (αGAL), N-glycolylneuraminic acid (Neu5Gc), and/or the SDa blood group (SDa) (e.g., Neu5Gc and SDa).

In some embodiments of the method described above, step (a) comprises contacting a sample obtained from the subject with ii) a test solid or semisolid support having immobilized thereon one or more Swine Leukocyte Antigens (SLA), and ii) a test solid or semisolid support having immobilized thereon one or more of galactose-α1,3-galactose (αGAL), N-glycolylneuraminic acid (Neu5Gc), and/or the SDa blood group (SDa) (e.g., Neu5Gc and SDa)

In some embodiments of the method described above, step (a) comprises contacting a sample obtained from the subject with i) a test solid or semisolid support having immobilized thereon cell membranes isolated from one or more porcine cells, wherein the porcine cells are isolated from the porcine donor or another animal within the same breeding family, ii) a test solid or semisolid support having immobilized thereon one or more Swine Leukocyte Antigens (SLA), and iii) a test solid or semisolid support having immobilized thereon one or more of galactose-α1,3-galactose (αGAL), N-glycolylneuraminic acid (Neu5Gc), and/or the SDa blood group (SDa) (e.g., Neu5Gc and SDa).

In various embodiments of the methods described above, the method further comprises contacting the sample obtained from the subject with
a negative control support comprising the same solid or semisolid support as in the test solid or semisolid support but without immobilized porcine cell membranes, and/or
a positive control support comprising the same solid or semisolid support as in the test solid or semisolid support with one or more immobilized porcine antigens known to cause a xenograft rejection response or react with IgM and/or IgG antibodies.

In some embodiments of the methods described above, the positive control support comprises one or more porcine antigens such as, but not limited to, galactose-α1,3-galactose (αGAL), N-glycolylneuraminic acid (Neu5Gc) and the SDa blood group (SDa). In one embodiment, the positive control support comprises porcine antigen galactose-α1,3-galactose (αGAL).

In some embodiments of the methods described above, the negative control sample does not have anti-porcine IgG and IgM antibodies. In some embodiments, the negative control sample does not have anti-human leukocyte antigen (HLA) antibodies.

In some embodiments of the methods described above, the dilution is prepared by diluting the sample with a solvent, such as, but not limited to, a saline solution or distilled water.

In some embodiments of the methods described above, the method further comprises proceeding with the xenograft transplantation from the porcine donor, when the subject is determined to be at a low risk of developing the xenograft rejection response.

In some embodiments of the methods described above, the method further comprises performing a treatment to reduce the amount of anti-porcine IgG and IgM antibodies in the blood of the subject prior to performing the xenograft transplantation from the porcine donor, when the subject is determined to be at a moderate or high risk of developing the xenograft rejection response.

In some embodiments, the treatment comprises one or more cycles of a plasma exchange. In some embodiments of the method described above, the method further comprises repeating steps (a)-(c) on the subject sample after said one or more cycles of the plasma exchange to determine if additional cycles of plasma exchange are needed prior to the xenograft transplantation. In some embodiments, the treatment comprises removing plasma cells and/or B cells from the blood of the subject.

In another aspect, the present disclosure provides a method of monitoring the risk of a subject developing a xenograft rejection response, wherein the subject has received a xenograft transplantation from a porcine donor, said method comprising:
a) contacting a sample obtained from the subject shortly before the transplantation and two or more samples obtained from the subject at different time points after the transplantation with a test solid or semisolid support having immobilized thereon cell membranes isolated from one or more porcine cells, wherein the porcine cells are isolated from the porcine donor or another animal within the same breeding family, or ii) a test solid or semisolid support having immobilized thereon one or more Swine Leukocyte Antigens (SLA), and/or iii) a test solid or semisolid support having immobilized thereon one or more of galactose-α1,3-galactose (αGAL), N-glycolylneuraminic acid (Neu5Gc) (any known variant and subtypes), and/or the SDa blood group (SDa) (any known variant and subtypes).
b) determining the amount of IgG and IgM antibodies bound to the test solid or semisolid support(s) for each of the samples used in step (a);
c) comparing the antibody amounts determined in step (b), and
d) determining that
i) the subject has a decreased risk of developing a xenograft rejection response when the amount of IgG and IgM antibodies in the sample obtained at a later time point is lower than the amount of IgG and IgM antibodies in the sample obtained at an earlier time point and the sample obtained shortly before the transplantation;
ii) the subject has an unchanged risk of developing a xenograft rejection response when the amount of IgG and IgM antibodies in the sample obtained at a later time point is the same as the amount of IgG and IgM antibodies in the sample obtained at an earlier time point and the sample obtained shortly before the transplantation, or
iii) the subject has an increased risk of developing the xenograft rejection response when the amount of IgG and IgM antibodies in the sample obtained at a later time point is higher than the amount of IgG and IgM antibodies in the sample obtained at an earlier time point and the sample obtained shortly before the transplantation.

In some embodiments of the method described above, the method comprises dilution of the subject sample in a solvent, e.g., from 1:2 to 1:32. In some embodiments, the solvent is a saline solution or distilled water. In some embodiments, the dilution is performed prior to step (a).

In some embodiments of the method described above, the method further comprises performing a treatment to reduce the amount of anti-porcine IgG and IgM antibodies in the blood of the subject, when the subject is determined to have an increased risk of developing the xenograft rejection response. In some embodiments, the treatment comprises one or more cycles of a plasma exchange. In some embodiments, the method further comprises repeating steps (a)-(d) on the subject sample after said one or more cycles of the plasma exchange to determine if additional cycles of plasma exchange are needed. In some embodiments, the treatment comprises removing plasma cells and/or B cells from the blood of the subject.

In some embodiments, a sample can be obtained about 1 week, about 6 days, about 5 days, about 4 days, about 3 days, about 2 days, about 1 day, about 18 hours, about 12 hours, about 6 hours, or about 4 hours, or about 2 hours, or about 1 hour before the transplantation.

In various embodiments of any one of the methods described above, the xenograft rejection response is an acute xenograft rejection response. For example, the acute xenograft rejection response may be an acute vascular rejection, hyperacute xenograft rejection, acute humoral xenograft rejection, and/or acute cellular rejection.

In various embodiments of any one of the methods described above, the xenograft rejection response is a delayed xenograft rejection response.

In another aspect, the present disclosure provides a method of selecting a porcine donor for xenograft transplantation for a subject in need thereof, said method comprising:
a) contacting a sample obtained from the subject with a plurality of differentially labeled solid or semisolid supports, each support having immobilized thereon cell membranes isolated from one or more cells of a single porcine donor from a plurality of candidate porcine donors or another animal within the same breeding family;
b) determining the amount of IgG and IgM antibodies bound to each of the
c) comparing the amounts of IgG and IgM antibodies determined in step (b) between different solid or semisolid supports, and
d) identifying a porcine donor for xenograft transplantation as the donor associated with the solid or semisolid support resulting in the lowest amount of IgG and IgM antibodies among the compared differentially labeled solid or semisolid supports.

In some embodiments of the method described above, the method further comprises proceeding with the xenograft transplantation using the identified porcine donor.

In various embodiments of any one of the methods described above, the xenograft transplantation is a transplantation of a whole organ or a tissue or cellular graft.

In various embodiments of any one of the methods described above, the amount of IgG and IgM antibodies is determined by contacting the solid or semisolid support, after exposure to the sample, with one or more labeled detection probes which recognize the IgG and/or IgM antibodies produced by the subject, and measuring a signal produced by the label. In some embodiments, the detection probe is an antibody which recognizes the IgG and/or IgM antibodies produced by the subject. In some embodiments, the label is a fluorescent dye or mediates a chemical reaction. In some embodiments, the label mediates an enzymatic reaction.

In another aspect, the present disclosure provides a method of identifying a new porcine antigen(s) recognized by anti-porcine antibodies in a subject, which antibodies mediate a xenograft rejection response in said subject to a xenograft transplantation from a porcine donor, said method comprising:
a) contacting a sample obtained from the subject with a solid or semisolid support having immobilized thereon cell membranes isolated from one or more porcine cells, wherein the porcine cells are isolated from the porcine donor or another animal within the same breeding family;
b) isolating IgG and/or IgM antibodies bound to the solid or semisolid support; and
c) determining target antigen(s) for the IgG and/or IgM antibodies isolated in step (b).

In another aspect, the present disclosure provides a method for creating a porcine xenograft donor with a minimized likelihood of inducing a xenograft rejection response, wherein the porcine donor is genetically modified to delete or inhibit the expression of the gene(s) encoding the porcine antigen(s) identified by the method described above.

In various embodiments of any one of the methods described above, the sample is selected from serum, plasma and an immunoglobulin fraction of the blood comprising IgG and IgM immunoglobulins.

In various embodiments of any one of the methods described above, the animal within the same breeding family is a parent of the porcine donor.

In various embodiments of any one of the methods described above, the porcine donor may be a porcine with α1,3-galactosiltransferase knock out (GTKO), a porcine with αGAL/SDa/Neu5Gc triple-knockout of α1,3-galactosiltransferase, CMP-N-acetylneuraminic acid hydroxylase and beta-1,4-N-acetyl-galactosaminyltransferase 2 (TKO), or a porcine with 10 genes modifications (10GE).

In various embodiments of any one of the methods described above, the one or more porcine cells may be one or more lymphocytes, endothelial cells, or epithelial cells, or a combination thereof. In some embodiments, the lymphocytes are T cells and/or B cells. In some embodiments, the endothelial cells are aortic endothelial cells. Other cell types may also be used, such as those described in Dash et al., Journal of Controlled Release 327 (2020) 546-570, which is incorporated herein by reference in its entirety.

Examples of solid support that can be used in various embodiments of any one of the methods described above include, but are not limited to, a bead, a particle, a microsphere, a microparticle, a plate, a microplate, a microtiter plate, a slide, a dish (e.g., a petri dish), a cup, a strand, a chip, a microchip, a strip, a membrane, a microarray, or a test tube. In some embodiments, the solid support is a bead. In some embodiments, the bead is a magnetic bead, plastic bead, microbead, polymer bead, or solid core bead.

In some embodiments, the semisolid or solid support may be a nanoparticle. In some embodiments, the semisolid support is a micelle. Suitable techniques that can be used to coat cell membranes to nanoparticles are described in, for example, Fang et al., Adv Mater. 2018 June; 30 (23): e1706759, which is herein incorporated by reference in its entirety.

In various embodiments of any one of the methods described above, the method is conducted in a high throughput format. In some embodiments, the method is conducted in a multi-well plate.

In various embodiments of any one of the methods described above, the subject is human.

In another aspect, the present disclosure provides a solid or semisolid support comprising immobilized thereon cell membranes isolated from one or more porcine cells. In some embodiments, the one or more porcine cells are one or more lymphocytes, endothelial cells, or epithelial cells, or a combination thereof. In some embodiments, the lymphocytes are T cells and/or B cells. In some embodiments, the endothelial cells are aortic endothelial cells. In some embodiments, the porcine cells are isolated from a porcine with α1,3-galactosiltransferase knock out (GTKO), a porcine with αGAL/SDa/Neu5Gc triple-knockout of α1,3-galactosiltransferase, CMP-N-acetylneuraminic acid hydroxylase and beta-1,4-N-acetyl-galactosaminyltransferase 2 (TKO), or a porcine with 10 genes modifications (10GE).

In another aspect, the present disclosure provides a solid or semisolid support comprising immobilized thereon one or more Swine Leukocyte Antigens (SLA).

In another aspect, the present disclosure provides a solid or semisolid support comprising immobilized thereon one or more galactose-α1,3-galactose (αGAL), N-glycolylneuraminic acid (Neu5Gc), and/or the SDa blood group (Sda).

In another aspect, the present disclosure provides a solid or semisolid support comprising immobilized thereon i) cell membranes isolated from one or more porcine cells, or ii) one or more Swine Leukocyte Antigens (SLA), and/or iii) one or more galactose-1,3-galactose (αGAL), N-glycolylneuraminic acid (Neu5Gc), and/or the SDa blood group (Sda).

In some embodiments of the solid or semisolid support described above, the solid support may be a bead, a particle, a microparticle, a plate, a microplate, a microtiter plate, a slide, a dish (e.g., a petri dish), a cup, a strand, a chip, a microchip, a strip, a membrane, a microarray, or a test tube. In some embodiments, the solid support is a bead. In some embodiments, the bead is a magnetic bead, microbead, polymer bead, or solid core bead. In some embodiments, the solid or semisolid sup-port is a nanoparticle. In some embodiments, the semisolid support is a micelle.

In some embodiments of the solid support described above, the solid or semisolid support is comprised within a multi-well plate.

In another aspect, the present disclosure provides a kit comprising
(i) the solid or semisolid support as described above;
(ii) optionally, a positive control support comprising the same solid or semisolid support as in the test solid or semisolid support with one or more immobilized porcine antigens known to cause a xenograft rejection response or react with IgM and/or IgG antibodies;
(iii) optionally, a negative control support comprising the same solid or semisolid support as in the test solid or semisolid support but without immobilized porcine cell membranes;
(iv) optionally, a labeled detection probe which recognizes IgG and/or IgM antibodies; and
(v) optionally, packaging and/or instructions for using the same.

In some embodiments, the one or more porcine antigens are selected from galactose-α1,3-galactose (αGAL), N-glycolylneuraminic acid (Neu5Gc) and the SDa blood group (SDa).

In some embodiments, the labeled detection probe is an antibody. In some embodiments, the label is a fluorescent dye or mediates a chemical reaction. In some embodiments, the label mediates an enzymatic reaction.

### BRIEF DESCRIPTION OF THE DRAWINGS

FIGS. 1A-1C show a comparison of IgG and IgM binding reactivity against GTKO and TKO as measured by flow crossmatch on pig lymphocytes (FIG. 1A) or complement-dependent cytotoxicity on pig aortic endothelial cells (pAECs) (FIGS. 1B-1C).

FIGS. 2A-2D show a comparison of IgG and IgM binding reactivity against GTKO. TKO and 10GE as measured by flow cytometry crossmatch (FCXM) on pig lymphocytes (FIG. 2A) or complement-dependent cytotoxicity assay (CDCXM) on pig aortic endothelial cells (FIGS. 2B-2D).

FIGS. 3A-3C illustrates the results of the first in-vivo immunological findings in human xenotransplantation. FIG. 3A shows the correlation between anti-pig IgM MCS and CDCXM for recipients 1 and 2. Recipient 2 expresses high titer anti-pig IgM Abs and tested positive by CDCXM on pAEC (FIG. 3A). FIGS. 3B-3C show 48 hours IgM IF staining (FIG. 3B) and C4d (FIG. 3C) of the transplanted porcine kidneys.

FIG. 4 shows the effect of plasma exchange (PLEX) on human IgG anti-pig antibodies. The IgG FCXM binding reactivity of this human serum, against GTKO pig lymphocytes, was reduced by 41% (from +967 to +575 MCS) with only one 1.5V PLEX.

FIG. 5 summarizes the essential steps of an exemplary solid-phase antibody detection assay. In solid-phase assay, the cell membrane of specific target cells is stripped and coated to plastic beads. These beads are then used as the target for the detection of antibodies.

FIG. 6 shows the correlation between serum reactivity and transplant risk in an exemplary method of the present disclosure. This classification allows to score human sera according to one of the following risk classifications: no risk (negative sera), weak risk (weakly positive sera), moderate risk (moderately positive sera), or high risk (strongly positive sera).

FIGS. 7A-7B show representations of the change in antibody titer following plasma exchange. Following PLEX, and based on the number of PLEX procedures, the titer of positive sera can be significantly reduced. FIG. 7A shows that a serum with moderate expression of pig-antibodies can be converted into a low/no risk by one or more PLEX. Similarly, a serum with strong reactivity to pig antibodies (FIG. 7B) can be reduced to moderate or low risk.

FIG. 8 is an exemplary flowchart illustrating possible decisions that can be made based on the outcomes of the disclosed solid-phase assays expressing different pig antigens.

### DETAILED DESCRIPTION

### Definitions

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

As used herein, the terms “about” or “approximately” for any numerical values or ranges indicate a suitable dimensional tolerance that allows the part or collection of components to function for its intended purpose as described herein. More specifically. “about” or “approximately” may refer to the range of values±20% of the recited value, e.g. “about 90%” may refer to the range of values from 71% to 99%.

The term “sample” as used herein includes any biological specimen obtained from a subject or patient. Samples that can be used in the methods of the present disclosure include, without limitation, serum, plasma, an immunoglobulin fraction of the blood, whole blood, red blood cells, white blood cells (e.g., peripheral blood mononuclear cells (PBMC), polymorphonuclear (PMN) cells, ductal lavage fluid, nipple aspirate, lymph (e.g., disseminated tumor cells of the lymph node), bone marrow aspirate, saliva, urine, stool (i.e., feces), sputum, bronchial lavage fluid, tears, fine needle aspirate (e.g., harvested by random periareolar fine needle aspiration), tumor sample, any other bodily fluid, a tissue sample such as a biopsy of a site of the tumor (e.g., needle biopsy), and cellular extracts thereof. In some embodiments, a sample described herein comprises serum, plasma or an immunoglobulin fraction of the blood comprising IgG and IgM immunoglobulins.

As used herein, the term “subject” or “patient” refers to mammals and includes, without limitation, human and veterinary animals. In a preferred embodiment, the subject is human.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an.” and “the” do not denote a limitation of quantity, but rather denote the presence of “at least one” of the referenced item.

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

### Solid-Phase Assays and Uses Thereof

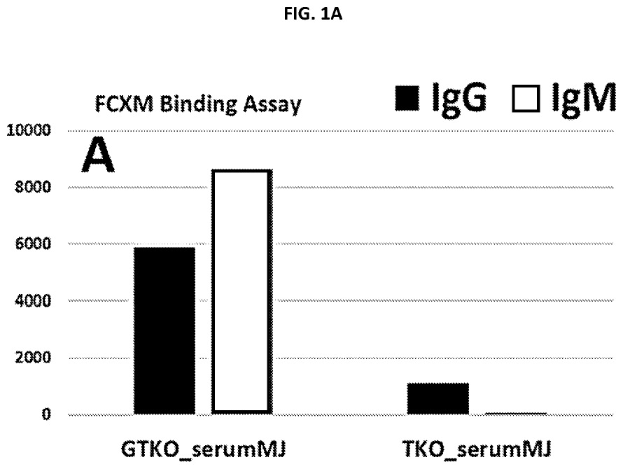

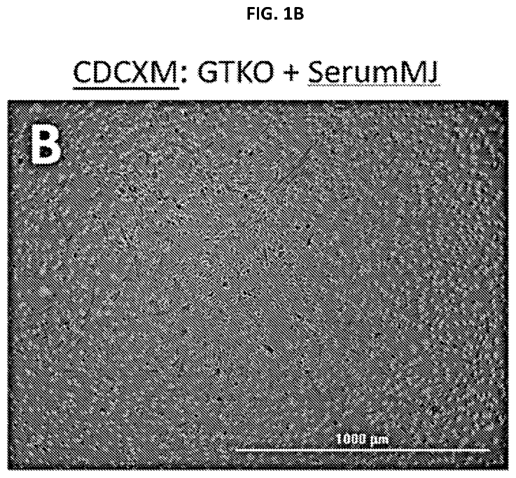

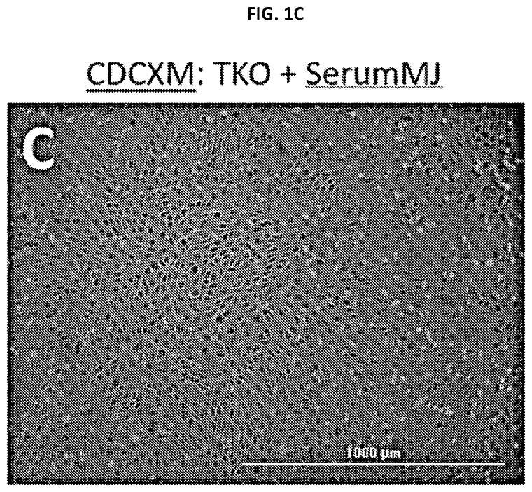

About 1% of all the antibodies present in human sera are directed against the galactose-α1,3-galactose (αGAL)^{2,3}, and αGAL epitopes are a major xenoantigen and a barrier to xenotransplantation⁴. For this reason, the α1,3-galactosiltransferase, the enzyme responsible for binding αGAL epitopes to cell surface proteins and lipids, has been targeted for disruption⁵. The generation of knockout pigs not expressing αGAL epitopes (GTKO) successfully prevented hyperacute rejection in both animal^{6,7} and human⁸ models. However, it become evident that naturally occurring antibodies against non-GAL antigens could cause a delayed type of rejection, termed “acute vascular rejection”⁹. This type of delayed xenograft rejection occurs over days or week, rather than in hours such as in the case for hyperacute rejection. Researchers were able to identify two additional xenogenic epitopes: SDa and Neu5Gc¹⁰. In particular, Neu5Gc is highly expressed on endothelial cells of all mammals with the exclusion of humans. Therefore, all human sera express a discrete amount of naturally occurring anti-Neu5Gc antibodies^{1,10}. The data presented herein (FIGS. 1A-1C) further indicate that human also express IgM and/or IgG antibodies targeting other (unknown) xenoantigens.

The assays described in the present disclosure (termed “PIGScreen assays”) allows, not only for the determination of the presence of anti-pig antibodies (e.g., lgG, IgM), but also the estimation of their clinical relevance to xenograft rejection.

Typically, there are at least three phases in transplantation: pre-transplant workup, crossmatch and antibody detection at time of transplant and post-transplant donor-specific antibody monitoring.

During pre-transplant workup, patients are screened for the presence of donor-specific antibodies. Currently, the only testing methods available for IgG/IgM detection on pig cells are the Flow Cytometry Crossmatch (FCXM) and complement-dependent cytotoxicity assay (CDCXM). Both rely on fresh cells and are not suitable methods for rapid and large-scale testing of potential xenotransplant recipients. For prospective, large-scale testing of human sera, the solid-phase PIGScreen assays can allow rapid and precise detection of anti-pig IgG and IgM antibodies. In addition to the known xenoantigens (e.g., αGAL, SDa and Neu5Gc), the T cell membrane provides Class I Swine Leukocyte Antigens (SLA) and B cells provide SLA Class II. Therefore, in some embodiments of the present disclosure, T cells and B cells are used as the source of cell membranes. T/B cell beads can account for cross-reactivity between HLA antibodies and SLA antigens¹⁶. This is important among individuals who have pre-formed anti-HLA antibodies due to previous sensitizing events. Additionally, pig aortic endothelial cells (pAECs) provide a source of non-lymphocyte xenoantigens that is specific to endothelial cells and potentially not present in lymphocytes, or have different levels of expression, and both tests together provide a more accurate quantitation of risk. Finally, because the assays described herein do not depend on fresh pig cells, they can be used to test multiple samples (e.g., 94 sera) at once. Therefore, the PIGScreen assays can be used as a novel screening tool for xenotransplant candidates.

At the time of transplant, a current serum (e.g., collected within 24 hours from transplant) from the intended recipient can be tested by both the solid-phase PIGScreen assay(s) and, when possible, by FCXM. Testing a current serum can ensure that there are no significant changes in sensitization from the historical sera.

Determining the current level of anti-pig antibodies pre-transplant is of extreme importance, since antibodies can be decreased below a putative clinical risk by means of plasma exchange (PLEX)¹⁷, as well as other highly effective methods of removing antibodies undergoing phase I, II, and III clinical trials.

Concurrent with organ transplantation, an FCXM on lymphocytes can be performed to determine the strength of IgG/IgM reactivity directly on the cells of the pig donor. This test is performed to confirm that the reactivity observed by the solid-phase PIGScreen assays is similar to the reactivity observed on donor lymphocytes (cell-based assay).

Post-transplant, patients can be monitored longitudinally for changes in IgG and/or IgM anti-pig antibodies. For a patient with pre-formed anti-pig antibodies, it is important to determine if an increase in reactivity is occurring, to appropriately intervene to reduce the antibody burden to prevent or reverse antibody mediated rejection. In unsensitized patients, it is important to determine if both an increase in preformed sensitization and/or a de-novo allo-sensitization caused by cross-reactivity with neo-antigens is occurring.

Solid-phase antibody assays are a very valuable resource to determine the reactivity against an antigen in a cell-free manner. Fresh cells are hard to obtain and have a very short half-life. Freezing target cells provides an alternative to freshly collected cells, but the process of thawing always results in cell loss and changes in the antigen's milieu in the cell surface. Eventually, every laboratory will have a shortage of frozen-stored cells and will need to procure more. Solid-phase assays that “mimic” target cells or express the antigens of target cells can solve the issue of being dependent on freshly collected cells.

Solid-phase assays are based on labeled solid or semisolid supports such as microspheres or beads about the size of a human lymphocyte. In some embodiments of the present PIGScreen assays described herein, membranes from pig cells, such as lymphocytes and endothelial cells, are striped and immobilized to plastic beads. The beads provide the solid support to conjugate specific antigens or the whole cell membrane content. The PIGScreen assay can include beads coated with a specific xenoantigen (e.g., a Swine Leukocyte Antigen (SLA), αGAL, Neu5Gc, or SDa), beads coated with the membrane from T and B cells, and beads coated with the membrane from pAEC. The beads coated by pig's cell membranes are then incubated with the target serum. If anti-pig antibodies are present, they will bind to the intended target and bound antibodies can be detected directly or by means of a secondary detection probe, e.g., a secondary antibody conjugated with a fluorochrome. The intensity of the reaction can be measured as emission wavelength in the logarithmic scale of the fluorochrome and assigned as weak, moderate, and strong.

Alternatively, the secondary detection probe may be conjugated to a label that is capable of mediating a chemical reaction (e.g., enzymatic reaction) to yield a signal. Examples of such labels include, but are not limited to, horseradish peroxidase (HRP), alkaline phosphatase (AP), β-galactosidase, acetylcholinesterase, and catalase. The choice of substrate depends upon the required assay sensitivity and the instrumentation available for signal-detection (e.g., spectrophotometer, fluorometer, or luminometer).

In one embodiment, Luminex® XMAP® beads (luminexcorp.com/xmap-technology/#overview) are used in the methods or compositions of the present disclosure. These beads are injected with different proportions of two dyes: red and infrared. This allows the creation of an array of up to 500 beads with different absorption/emission wavelengths. This array of beads can be used in a multiplex assay and detected by xMAP instruments or 2-lasers flow cytometers.

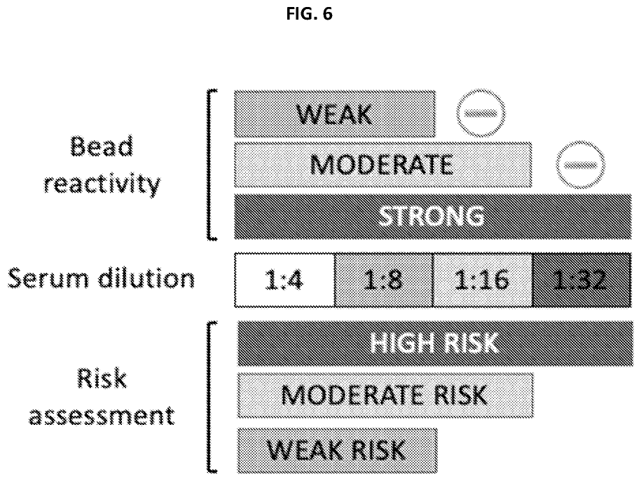

The result of the solid-phase PIGScreen assays and the risk classification (see, e.g., FIG. 6) represent a powerful diagnostic tool that can inform the surgical team about the potential risk for xenotransplant and allow for pre- and post-transplant individualized therapeutic intervention.

In the pre-transplant phase, transplant candidates can be tested for the presence and titer of anti-pig antibodies. This information has a strong diagnostic value and can be used to identify candidates at risk of early rejection.

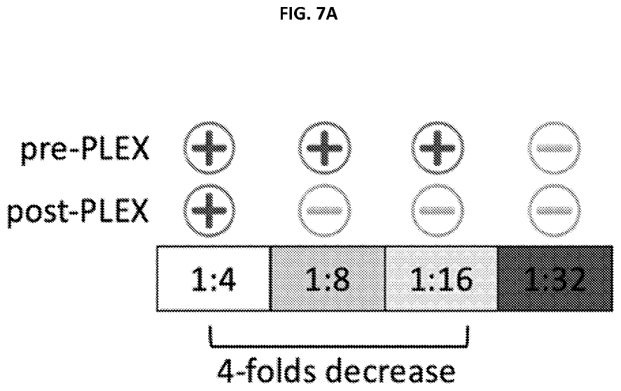

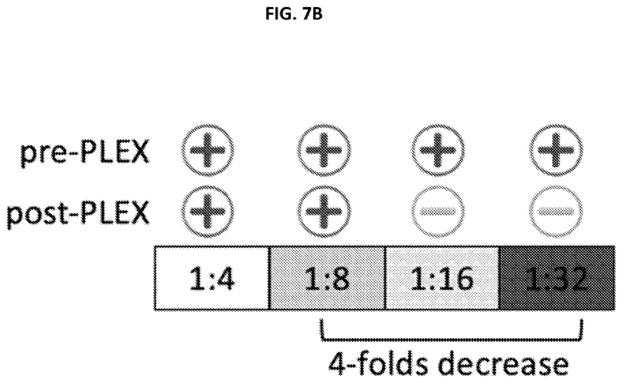

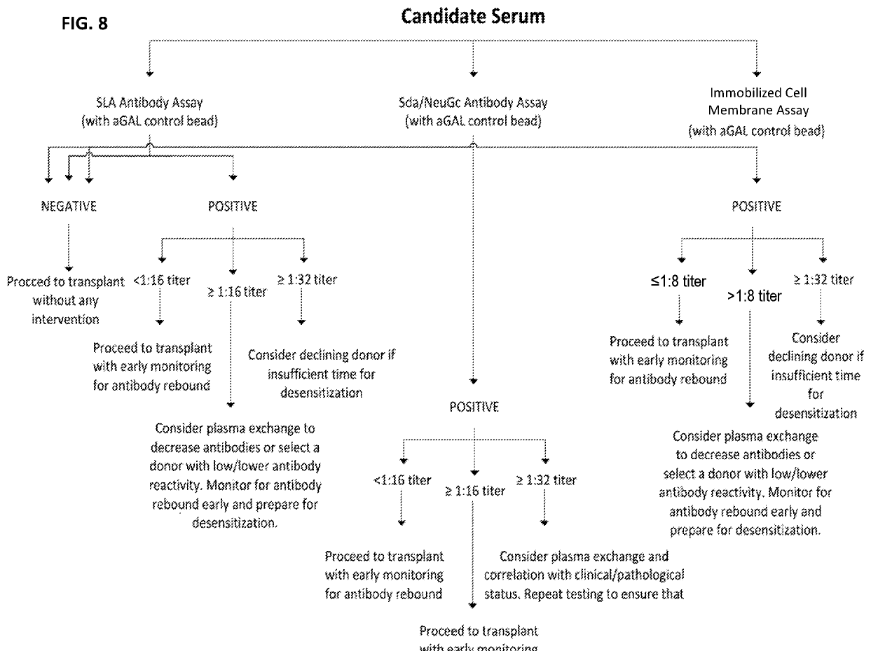

In fact, the higher the antibody titer, the more likely the patient will experience antibody-mediated damage of the xenograft and graft lost. For transplant candidates that are highly sensitized against human organ (high cPRA against human HLA antigens) and that have exhausted options for dialysis, xenotransplantation represents the only option. However, the concomitant presence of high-titer anti-pig antibodies may also prevent transplantation with pig organs. Therefore, for patients positive for the expression of pig-antibodies, therapeutic intervention with plasma exchange (PLEX) can reduce the antibody burden and facilitate xenotransplantation. By removing about 63.2% of IgG molecules²³, a single PLEX can significantly decrease the level of antibodies and prevent rejection. For examples, as show in FIG. 7, a serum with moderate expression pig-antibodies (FIG. 7A) can be converted into a low/no risk by one or more PLEX. Similarly, a serum with strong reactivity to pig antibodies (FIG. 7B), can be reduced to moderate or low risk. Based on the results from the PIGScreen assay, patients can be classified for risk of rejection. If a candidate is positive for the presence of anti-pig antibodies against any genetic construct and against multiple lineages of the same construct, PLEX can be used to intervene to reduce the antibodies. After each PLEX, the post-PLEX serum is re-tested, and its reactivity is compared to the reactivity in the pre-PLEX serum. This step can be repeated until the anti-pig antibodies demonstrate a low-titer (e.g., ≤1:8) reactivity.

After transplant, one of the most important diagnostic tools is serial monitoring of the level of anti-pig antibodies. In the post-transplant phase, at least two things can happen: increase of pre-formed antibodies and/or generation of new antibodies.

Transplant patients without anti-pig antibodies may develop new ones by means of de-novo sensitization to previously known or new xenoantigens. Patients with pre-formed anti-pig antibodies may increase the expression by means of memory response. In both cases, as shown in FIG. 7, therapeutic intervention with PLEX can be employed if the anti-pig antibodies reach a titer>1:16. Also in this case, comparison of sera reactivity pre- and post-PLEX can provide important diagnostic information and inform the medical team as to whether more intervention is needed. By routinely testing for the expression of anti-pig antibodies, the xeno-organ can be protected from the presence of high-titer organ-specific antibodies and shielded from humoral rejection.

Additionally, apart from antibodies against αGAL, SDa, and Neu5Gc, it is currently unknown which antibodies can be formed post-transplant. By coupling beads with intact membranes of both lymphocytes and endothelial cells, the solid-phase PIGScreen assays described herein provide a means to detect new antibodies not previously know. For a transplant patient previously negative for the presence of pig antibodies, this assay can provide a less invasive method for diagnostic detection of de-novo pig antibodies. If a transplant patient develops new anti-pig antibodies, early detection may represent the only chance at preventing rejection. If new antibodies are detected at a titer of, for examples, >1:16, intervention with PLEX can remove the antibody burden and prevent antibody-mediated rejection. Additionally, the detection of previously unknown antibodies, is a very important step in the translational research. New antibodies can be isolated, and their specificity can be investigated. This may lead to the discovery of new xenoantigens that can be ultimately target of genetic targeted disruption. Because cells from the pig donor are not available post-transplant, the PIGScreen assay can prove essential to both rejection prevention and translational research.

In addition to the advantages described above, the solid-phase PIGScreen assays also have a long shelf-life (about 12 months), the assays do not require cell culture and is easy to perform. Some embodiments of the present disclosure include a multiplexed assay (testing multiple “targets” in one reaction). The swine-antibody detection panel can include beads coated with the antigenic products of several different genetic pig constructs, thus allowing the clinical teams to test each patient serum against several pigs, simultaneously. The PIGScreen assays described herein allow transplant centers and their laboratories to quickly implement the assays without the need to obtain fresh blood from pig farms. Its multiplex capabilities allow centers to test multiple “options” in one reaction, for example, the assays can be run for 96 sera at the same time. The PIGScreen assays described herein allow advancement in xenotransplantation by making human anti-pig antibody detection an easy-to-use reality which can lead to a better understanding of the post-transplant significance of these antibodies.

### EXAMPLES

The following examples are provided to further describe some of the embodiments disclosed herein. The examples are intended to illustrate, not to limit, the disclosed embodiments.

### Example 1. Pre-Formed Anti-Pig Antibodies in Human Sera

Advancement in CRISPR technology allowed the generation of triple-knockout pigs (TKO) where, along with suppression of αGAL antigen, the expression of both SDa and Neu5Gc is inhibited¹¹. However, animal¹¹ and human (FIGS. 1A-1C) models both indicate the presence of IgM and/or IgG antibodies targeting other (unknown) xenoantigens.

FIG. 1A depicts the Flow Cytometry Crossmatch (FCXM) binding strength (median channel shift—MCS) of non-GAL anti-pig IgG and IgM antibodies expressed by a randomly selected human serum. The reduction in binding strength for both IgG and IgM against TKO lymphocytes is readily evident. Similarly, as compared to the reactivity against endothelial cells from a GTKO pig (FIG. 1B), the complement-dependent cytotoxicity assay (CDCXM) reactivity against pig Aortic Endothelial Cells (pAEC) from a TKO pig is also significantly reduced (FIG. 1C). Although the target of these non-GAL, −SDa and −Neu5Gc antibodies is not yet known, the question still remains if these antibodies can cause delayed xenograft dysfunction.

More recently, a genetically engineered pig construct with 10 genes modifications (10GE) has been introduced. The genetic modification of the 10GE line include 3 knockout endogenous genes (to inhibit the expression of αGAL. SDa, and Neu5Gc) and 6 knock-in (insertion of human genes) to inhibit the activation of human complement (hDAF, hCD46), prevent coagulation (hTBM, HEPCR) and to modulate the immune system functions (hCD47, hHO1). The 10GE construct was used to transplant a pig kidney to a human decedent and a pig heart to a living human recipient^{12, 13}.

To test the hypothesis that a 10GE pig construct is more favorable in terms of naturally occurring anti-pig antibodies, the same serum (serumMJ) was tested against 10GE pig lymphocytes and pAEC.

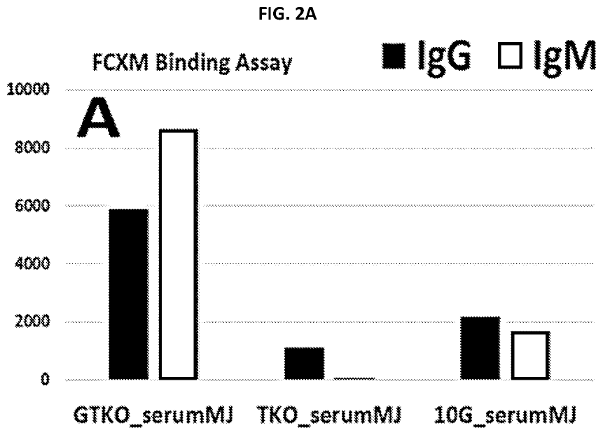

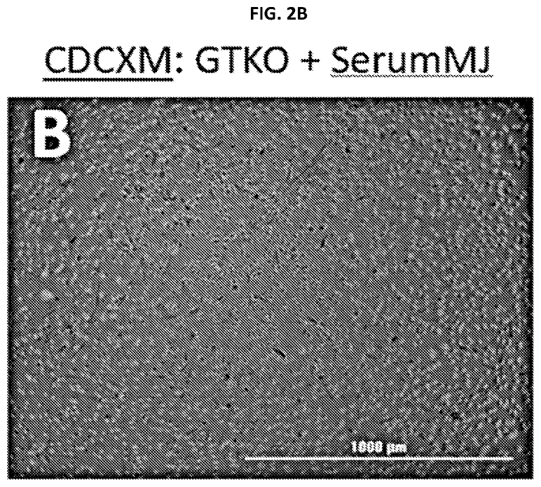

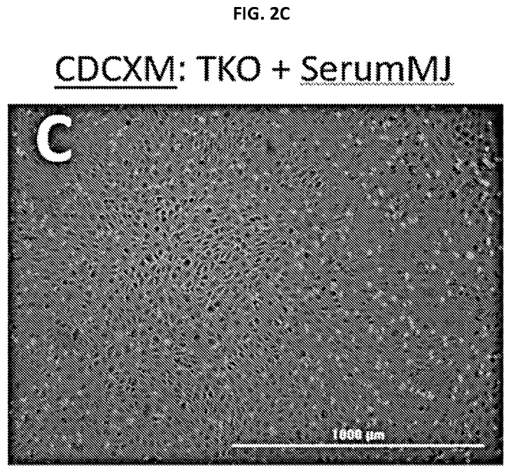

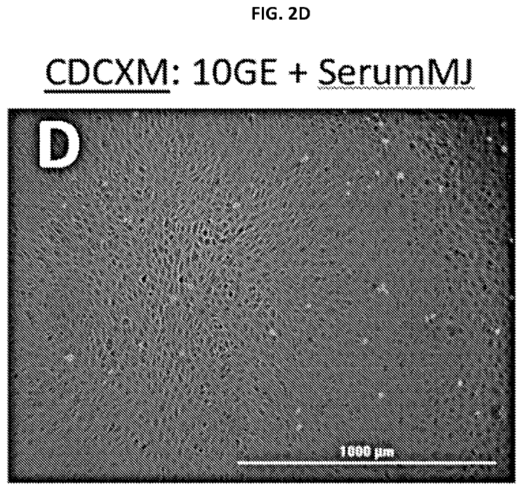

FIG. 2 shows the IgG/IgM flow binding reactivity on 10GE pig lymphocytes (FIG. 2A) is similar if not superior to that observed against TKO lymphocytes. However, the reactivity by CDCXM (FIG. 2D) is significantly lower than that observed on both GTKO (FIG. 2B) and TKO (FIG. 2C) pAEC. These results clearly demonstrate that the 10GE pig still express a discrete amount of xenoantigens, although their capability of activating the complement cascade is inhibited. Therefore, the only immediate advantage of the 10GE pig construct is the inhibition of the classical complement cascade. However, antibodies can still damage the organ by the mechanism of antibody-dependent cellular cytotoxicity (ADCC)¹⁴. Although the DAF and CD46 effectively inhibit the acute function of the anti-pig antibodies in the 10GE pigs, the presence of anti-pig antibodies in human sera is still dangerous and can lead to delayed graft dysfunction by ADCC. Therefore, detection of anti-pig antibody and measurement of their titer is essential to determine the risk of delayed xenograft functions.

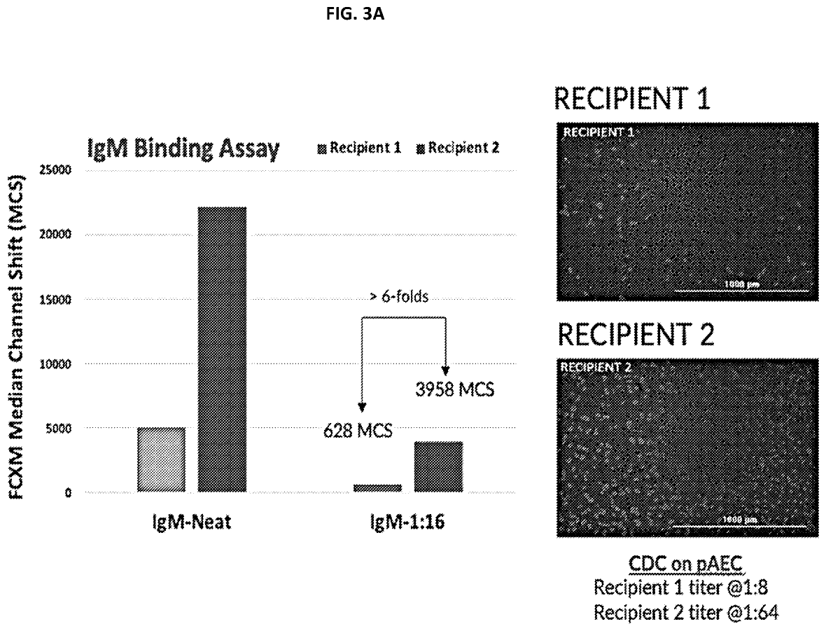

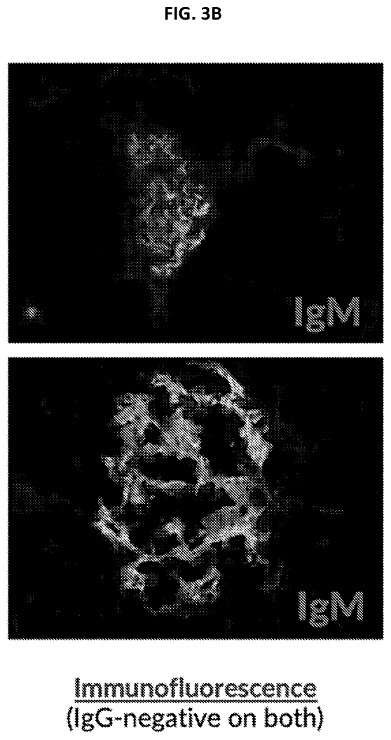

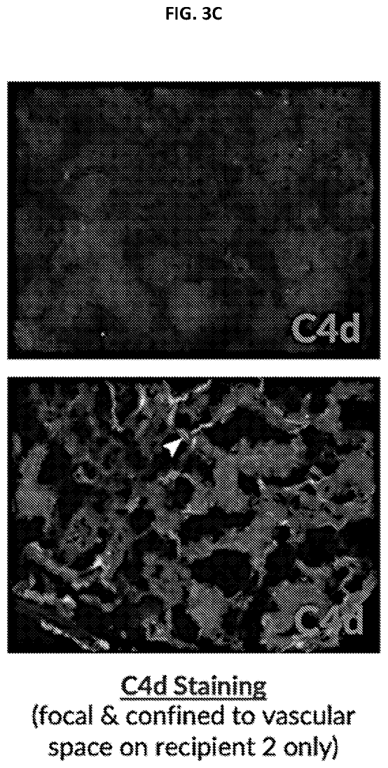

Recently, the first two GTKO pig kidney transplants were performed in decedent humans⁸. The GTKO kidneys were transplanted into two brain-dead human recipients whose cardiocirculatory functions were artificially maintained for the duration of the study. This study created the opportunity to determine for the first time the effect that human anti-pig antibodies have on kidney xenografts. The presence of non-GAL anti-pig IgG/Ig.V antibodies were tested by FCXM on pig lymphocytes before transplant and CDCXM on pAFC was tested retrospectively. During the study period, renal functions and xenograft rejection were monitored by serial biopsies, urine output and estimated glomerular filtration rate (eGFR). At the end of the study, the two xeno-kidneys were found to have no signs of hyperacute or antibody-mediated rejection and with normal clinical functions⁸. The only significant differences between the two xenografts were the presence of bound IgM in the vasculature of the glomeruli and focal C4d deposition in one but not both pig kidneys⁸. FIGS. 3A-3C summarize the results of the first in vivo immunological finding in human xenotransplantation¹⁵.

Recipient 2 had more IgG and more IgM anti-pig antibodies as demonstrated by neat (undiluted) serum and the 1:16 dilution (FIG. 3A). These high titer binding antibodies (especially the IgM subclass) were complement-activating, as demonstrated by the positive CDCXM on pAEC (FIG. 3A). In-vivo, the IgM binding was much stronger on recipient 2 and C4d staining was focally positive (FIG. 3B-3C), thus indicating that high titer anti-pig antibodies can induce complement activation in-vivo.

### Example 2. Cells Source and Membranes Isolation

Peripheral pig lymphocytes and pig aortic endothelial cells (pAEC) are purchased from Revivicor (Blacksburg, VA). Because lymphocytes have a lower density as compared to granulocytes and erythrocytes, isolation of pig peripheral blood mononuclear cells (PBMC) is achieved by means of density gradient media (ficol)¹⁸. After PBMC isolation, T and B cells can be isolated by means of magnetic beads of cell sorting based on the differential expression in SLA class II. Swine endothelial cells can be isolated from swine hearts or swine aortic arch and according to well established isolation, culturing, and expansion protocols¹⁹. To maintain a constant source of cells and membranes, T and B cell lymphocytes and pAEC are immortalized using procedures described in, for example, Kaeffer et al.²⁰ and Carrillo et al.²¹ (both of which are hereby incorporated by reference in their entireties). Preparation of cell membranes is performed by sequential isolation of cellular components based on their differential weight and density and according to the procedures as described in, for example, Allan and Crumpton²² (which is hereby incorporated by reference in its entirety). T cells, B cells and pAEC membranes are coupled with Luminex® XMAP® beads and initial quality control is performed. To validate the assay kit and establish the final standard operating procedure (SOP), testing on human sera and correlation with cell-based assay is performed.

### Example 3. Solid-Phase PIGScreen Assay With Cell Membrane-Coated Beads

In this Example, the solid-phase PIGScreen assay utilizes beads that are coated with pig's cell membranes, which “mimics” both pig lymphocytes and pig endothelial cells. Detection of IgG and/or IgM on the bead surface therefore mimics the detection of the same immunoglobulins, but on a lymphocyte or pAEC cell. Therefore, the PIGScreen assay is a very valuable replacement to a physical FCXM or CDCXM.

The assay comprises 3 populations of assay beads which are described in Table 1 below. The porcine T cells, B cells, and pAECs originate from three distinct genotypes: an αGAL knockout genotype (GTKO), a triple knockout genotype (TKO), and a 10 gene modification genotype (10GE). Because the Luminex® XMAP® technology (luminexcorp.com/xmap-technology/#overview) allows multiplexing, all the beads listed above have a different adsorption/emission spectrum, are assigned to different “luminex regions” and are therefore be run in the same test tube.

The following instruments and equipment are needed for this assay: a LABScan3D flow analyzer (Luminex® FLEXMAP 3D®) with XY platform and sheath fluid delivery system, a centrifuge, a Rotor for 1.5 ml microcentrifuge tube (9.300 g) or a swinging bucket rotor for 96-well microplate (1,300 g), a vortex mixer, and a plate shaker or a rotating platform. For filter plate option, a vacuum manifold, 96-well (Millipore Cat. #MAVM0960R or equivalent), a vacuum pump with a pressure less than 100 mm Hg, and a plate shaker or rotating platform are needed.

Unopened blood specimens may be kept at room temperature up to 4 days. Separated serum (from clotted samples) or plasma (in acid-citrate-phosphate (ACD) or potassium-ethylenediaminetetraacetic acid (K-EDTA)) may be refrigerated up to 7 days, or aliquots may be frozen at −20° C. or below and thawed just before the assay. Aggregates should be removed from the test serum/plasma by centrifugation (8.000-10,000 g for 10 minutes) or filtration (0.2 μm) prior to testing. Any aggregates or contamination of the sample may generate invalid results. Samples may be treated or diluted to reduce non-specific background or to remove inhibitory factor.

The following materials are needed for this assay: Luminex® XMAP® test beads coated with pig T/B lymphocytes and pAEC (further described in Table 1). Luminex® XMAP® positive control beads coated with α-GAL, SDa and NEU-Gc porcine antigens, Luminex® XMAP® negative beads not coated (naked beads), negative control serum (a serum negative for HLA and anti-pig antibodies), test serum (a serum from a xenotransplant candidate) including necessary serum dilutions, PE-conjugated goat anti-human IgG (OLI Cat. #LS-AB2). PE-conjugated goat anti-human IgM (OLI Cat. #IGM-PEC1), filtered PBS [USA Scientific Cat. #9242 (500 ml 10×) or equivalent], 1.5 ml microcentrifuge tubes (USA Scientific Cat. #1415-2500 or equivalent), and pipette tips.

For each assay reaction, two 1.5 ml microcentrifuge tubes are prepared by pre-labeling each tube with the test serum ID and identify which tube is to be used for IgG or IgM detection.

The procedure for this assay is as follows:

The PIGScreen beads are mixed well by gently vortexing or pipetting up and down several times prior to use. In each 1.5 ml micro-centrifuge tube. 5 μL of PIGScreen beads are incubated with 20 μL of test serum in a for 30 minutes, in the dark at 20-25° C. with gentle shaking, 10× wash buffer (OLI Cat. #LSPWABUF) is diluted in distilled water to make a 1× solution. 1 mL of 1× wash buffer is added to each bead/serum solution tube and vortex. Each tube is centrifuged at 9,300 g for 2 minutes. The supernatant of each tube is aspirated and discarded. The 1 mL buffer wash, the centrifugation, and the aspiration steps are repeated twice more. 1 μL of 100×PE-conjugated anti-human IgG (OLI Cat. #LS-AB2) and 100×PE-conjugated anti-human IgM (OLI Cat. #IGM-PEC1) are diluted with 99 μL of 1× wash buffer to make a 1× solution. 100 μL of 1× PE-conjugated anti-human IgG and IgM are added to each specific tube. Tubes are vortexed and then incubated in the dark for 30 minutes at 20-25° C. with gentle shaking. The 1 mL buffer wash, the centrifugation, and the aspiration steps are repeated twice more. 80 μL 1× PBS is added to each tube. Data acquisition and analysis are performed, or tubes are stored at 2-8° C. in the dark for up to 24 hours before analysis.

After data acquisition, serum risk and reactivity are scored based upon the criteria in FIG. 6. As shown in FIGS. 3A-3C, the antibody titer, more than the relative expression in the undiluted serum, is what appears to correlate with activation of complement and the potential for acute damage. The higher the anti-pig antibody titer, the more likely it is for the xenograft to experience complement activation and acute damage. Therefore, the reactivity of human sera against the PIGScreen beads can be qualified as:

### Negative

when test beads are ≤the negative bead

### Weak Reactivity

when test beads are negative at 1:16 dilution

### Moderate Reactivity

when test beads are negative at 1:32 dilution

### Strong Reactivity

when test beads are positive at 1:32 dilution.

As shown in FIG. 6, this classification allows the human sera to be scored according to the following risk classification:
NO RISK=negative sera
WEAK RISK=weak positive sera
MODERATE RISK=moderate positive sera.
HIGH RISK=strong positive sera.

For example, if a patient is positive at a 1:32 dilution, this indicates a positive signal for increased risk of activating a complement cascade, affecting xenobiotic organ transplantation. Correlation of such antibody titer to complement-dependent cytotoxicity crossmatch (CDCXM) has been shown in Mangiola M et al.¹⁵, which is incorporated herein by reference in its entirety.

TABLE 1
Categories of Beads Needed for PIGScreen Assay

| Negative | Beads are not coated with a biological |
|---|---|
| Control Beads | product (i.e., naked beads) |
| Positive | Beads coated with αGAL |
| Control Beads | Beads coated with SDa |
| | Beads coated with Neu5Gc |
| Test Beads | Beads coated with pig T cell membrane from a GTKO |
| | Beads coated with pig B cell membrane from a GTKO |
| | Beads coated with pAEC membrane from a GTKO |
| | Beads coated with pig T cell membrane from a TKO |
| | Beads coated with pig B cell membrane from a TKO |
| | Beads coated with pAEC membrane from a TKO |
| | Beads coated with pig T cell membrane from a 10GE |
| | Beads coated with pig B cell membrane from a 10GE |
| | Beads coated with pAEC membrane from a 10GE |

### Example 4. PIGSceen-Multi Assay

Because pig herds are not cloned but maintained by inbreeding, pigs from different genealogies may have unique xenoantigen phenotypes with unique expression levels. Like in human-to-human organ transplantation, identification of the best xeno-organ donor is essential to ensure the most favorable outcome. Although the expression of known and unknown xenoantigens may not differ significantly in pigs from the same germline, among germlines pigs may express different numbers of immunogenic antigens and this information is very important. In fact, initial observations on GTKO pigs does indicate that human sera with moderate IgG/IgM FCXM reactivity against SDa and Neu5Gc may significantly differ in some but not all pigs. Therefore, the PIGSceen Multi assay is a powerful diagnostic tool that allows for the identification of the best organ donor and therefore an individualized approach to xenotransplantation.

This assay utilizes beads coated with the membranes from GTKO, TKO and 10GE animals from multiple pig lineages. The assay comprises 3 populations of assay beads which are described in Table 2 below. The instruments, equipment, and serum handling in this example are identical to those described in Example 1.

The following materials are needed for this assay: Luminex® xMAP® test beads coated with pig T/B lymphocytes and pAEC (further described in Table 2). Luminex® xMAP® positive control beads coated with α-GAL, SDa and NEU-Gc porcine antigens, Luminex® xMAP® negative beads not coated (naked beads), negative control serum (a serum negative for HLA and anti-pig antibodies), test serum (a serum from a xenotransplant candidate) including necessary serum dilutions, PE-conjugated goat anti-human IgG (OLI Cat. #LS-AB2). PE-conjugated goat anti-human IgM (OLI Cat. #IGM-PEC1), filtered PBS [USA Scientific Cat. #9242 (500 ml 10×) or equivalent], 1.5 ml microcentrifuge tubes (USA Scientific Cat. #1415-2500 or equivalent), and pipette tips.

For each assay reaction, two 1.5 ml microcentrifuge tubes are prepared by pre-labeling each tube with the test serum ID and identify which tube is to be used for IgG or IgM detection. The procedure for this assay is as follows:

The PIGcreen-Multi beads are mixed well by gently vortexing or pipetting up and down several times prior to use. In each 1.5 ml micro-centrifuge tube, 5 μL of PIGScreen beads are incubated with 20 μL of test serum in a for 30 minutes, in the dark at 20-25° C. with gentle shaking. 10× wash buffer (OLI Cat. #LSPWABUF) is diluted in distilled water to make a 1× solution, 1 ml, of 1× wash buffer is added to each bead/serum solution tube and vortex. Each tube is centrifuged at 9.300 g for 2 minutes. The supernatant of each tube is aspirated and discarded. The 1 mL buffer wash, the centrifugation, and the aspiration steps are repeated twice more. 1 μL of 100×PE-conjugated anti-human IgG (OLI Cat. #LS-AB2) and 100×PE-conjugated anti-human IgM (OLI Cat. #IGM-PEC1) are diluted with 99 μl of 1× wash buffer to make a 1× solution. 100 μl of 1×PE-conjugated anti-human IgG and IgM are added to each specific tube. Tubes are vortexed and then incubated in the dark for 30 minutes at 20-25° C. with gentle shaking. The 1 mL buffer wash, the centrifugation, and the aspiration steps are repeated twice more. 80 μl 1×PBS are added to each tube. Data acquisition and analysis are performed, or tubes are stored at 2-8° C. in the dark for up to 24 hours before analysis.

After data acquisition, serum risk and reactivity are scored based upon the criteria in FIG. 6. For example, the reactivity of human sera against the PIGScreen beads can be qualified as:

### Negative

when test beads are ≤the negative bead

### Weak Reactivity

when test beads are negative at 1:16 dilution

### Moderate Reactivity

when test beads are negative at 1:32 dilution

### Strong Reactivity

when test beads are positive at 1:32 dilution.

The human sera can be classified according to the following risk classification:
NO RISK=negative sera
WEAK RISK=weak positive sera
MODERATE RISK=moderate positive sera
HIGH RISK=strong positive sera.

For example, if a patient is positive at a 1:32 dilution, this indicates a positive signal for increased risk of activating a complement cascade, affecting xenobiotic organ transplantation.

Based on the above information, the best organ donor can be identified.

TABLE 2
Categories of Beads Needed for PIGScreen Assay

| Negative | Beads are not coated with a biological |
|---|---|
| Control Beads | product (i.e., naked beads) |
| Positive | Beads coated with αGAL |
| Control Beads | Beads coated with SDa |
| | Beads coated with Neu5Gc |
| Test Beads | Beads coated with pig T cell membrane from a |
| | GTKO-Lineage 1 through 6 |
| | Beads coated with pig B cell membrane from a |
| | GTKO-Lineage 1 through 6 |
| | Beads coated with pAEC membrane from a GTKO- |
| | Lineage 1 through 6 |
| | Beads coated with pig T cell membrane from a |
| | TKO-Lineage 1 through 6 |
| | Beads coated with pig B cell membrane from a TKO- |
| | Lineage 1 through 6 |
| | Beads coated with pAEC membrane from a TKO- |
| | Lineage 1 through 6 |
| | Beads coated with pig T cell membrane from a |
| | 10GE-Lineage 1 through 6 |
| | Beads coated with pig B cell membrane from a |
| | 10GE-Lineage 1 through 6 |
| | Beads coated with pAEC membrane from a 10GE- |
| | Lineage 1 through 6 |

### Example 5. PIGScreen Assay from Pre-to Post-transplant

The assay materials, instruments, and procedure in this example are identical to those described in Example 1. In the pre-transplant phase, patients are screened for the presence of anti-pig antibodies and monitored with multiple assays performed over time, up to transplant day. Post-transplant, patients are screened to determine if the anti-pig antibodies are increasing, which may be indicative of a humoral rejection response. These assays provide information about the relative amount of anti-pig antibodies and can be used to determine the efficacy of antibody desensitization strategies.

### Example 6. Antibody Desensitization by Plasma Exchange

If a candidate is positive for the presence of anti-pig antibodies against any genetic construct and against multiple lineages of the same construct, plasma exchange (PLEX) can be used to reduce the antibodies. After each PLEX, the post-PLEX serum is re-tested, and its reactivity is compared to the reactivity in the pre-PLEX serum. This step is repeated until the anti-pig antibodies demonstrate a low-titer (e.g., ≤1:8) reactivity.

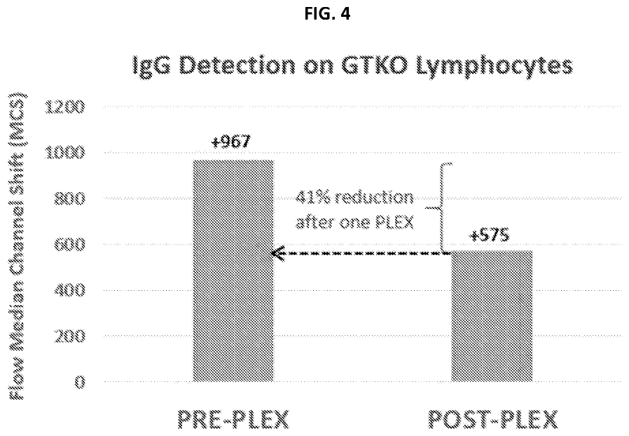

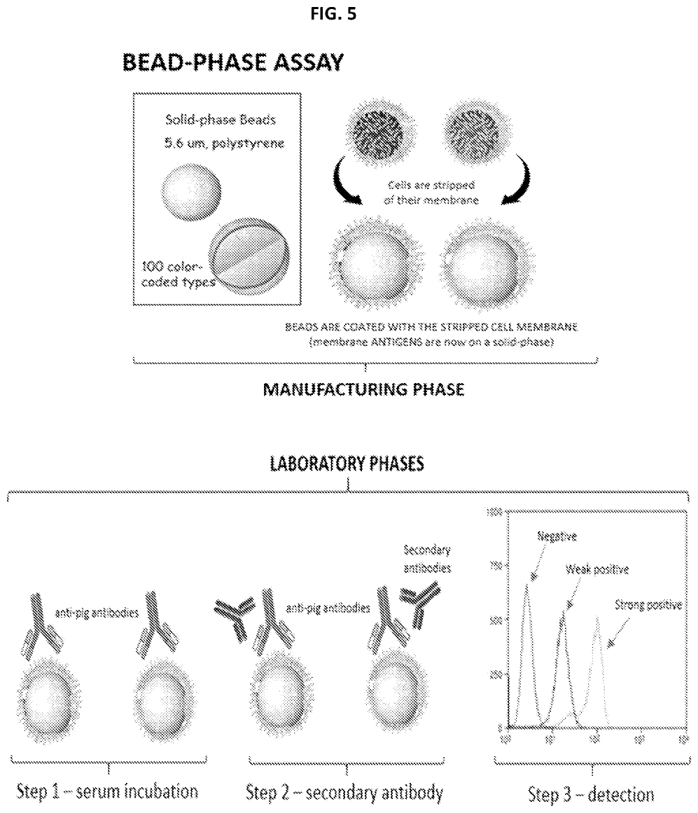

As shown in FIG. 4, the IgG binding strength of a randomly selected human serum against GTKO pig lymphocytes was successfully decreased by about 41% with only one plasma exchange. This proves that anti-pig antibodies can be efficiently decreased with PLEX. Therefore, therapeutic plasma exchange (TPE) can be used to desensitize xenotransplant candidates, when their naturally occurring antibodies are a contraindication to proceed to transplantation. Desensitization by PLEX allows also rescue from memory recall or de-novo allo-sensitization post-transplant, thus providing a therapeutic intervention option that can prevent graft dysfunction and loss.

### Example 7. Solid-Phase PIGscreen Assays Expressing Different Pig Antigens

Human sera are known to express naturally occurring antibodies against xeno-antigens which can cause hyperacute, acute or accelerated rejection. Human sera are known to express anti-galactose-α1,3-galactose (αGAL) antibodies, and most express a discrete amount of both anti-SDa glycan and anti-N-glycolylneuraminic acid (Neu5Gc) antibodies. Additionally, because of the homology between Human Leukocyte Antigens (HLA) and Swine Leukocyte Antigens (SLA), a concern exists as to whether HLA antibodies may cross-react with SLA antigens and cause rejection^{16,27}.

Currently a method to detect these antibodies individually does not exist. Knowing which antibody is present and at which titer has a significant weight in pre- and post-transplant patient management.

The generation of solid-phase assays that allow for the detection and characterization of individual anti-pig antibodies will facilitate patient management and potentially improve organ survival. Distinguishing naturally occurring anti-carbohydrates antibodies from antibodies targeting protein molecules is of clinical significance. Anti-carbohydrate antibodies are usually low affinity and low avidity antibodies whereas anti-protein antibodies are usually high affinity and high avidity²⁸. Carbohydrate antibodies are generally classified as derived from T-cell independent antigen presentation, which results in B cell responses (antibodies) that are lacking affinity maturation toward the antigen and primarily the production of IgM and IgG2 antibodies²⁸. Similar to anti-ABO antibodies, anti-pig antibodies targeting carbohydrates antigens are titer-dependent and will not cause Antibody-Mediated Rejection (AMR) if their titer is ≤1:8 at the time of transplant^{28,29}. Post-transplant, anti-carbohydrate antibodies do not seem to cause rejection if their titer does not exceed 1:64 and if the endothelial cells activate the mechanism of accommodation³⁰. Conversely, antibodies targeting protein epitopes are generally classified as derived from T-cell dependent antigen presentation, which results in B cell responses that produce antibodies with high affinity maturation and high avidity²⁸. High affinity antibodies generated from mismatched HLA proteins are directly correlated with allograft rejection and pre-formed HLA antibodies are a significant contraindication to transplantation³¹.

Therefore, in complex sera expressing a combination of naturally occurring low affinity antibodies and high affinity antibodies derived from an adaptive immune response, the ability to distinguish which antibody is present and their individual titer is of high significance in transplantation.

### Solid-Phase Assay for the Detection of Anti-αGAL Antibodies

A large proportion of naturally occurring antibodies are directed against the αGAL epitope. A solid-phase assay for the detection of αGAL antibodies would primarily be used as internal positive control.

### Solid-Phase Assay for the Detection of Anti-SDa and Anti-Neu5Gc Antibodies

Antibodies against SDa and Neu5Gc could pose a significant risk for acute or accelerated rejection if these antibodies have a titer≥1:16. During the pre-transplant phase, a SDa/Neu5Gc-specific assay would inform the clinical team about the titer strength of these antibodies and be used as indication for therapeutic plasma exchange (PLEX). During the post-transplant phase, a rise in these antibodies to a titer≥1:32 in conjunction with a rise in creatinine level and a decrease in eGFR, would trigger intervention with PLEX or explorative biopsy.

### Solid-Phase Assay for the Detection of Anti-SLA Antibodies

SLA are protein expressed by all nucleated pig cells. The presence of anti-SLA antibodies would indicate that a T cell-dependent adaptive immune process took place and that memory T cells, memory B cells, long lived plasma cells and high affinity IgG antibodies are present. Low and high titer anti-SLA antibodies pose a risk for delayed rejection or acute/accelerated rejection, respectively. At the time of pre-transplant evaluation, detection and titer of SLA antibodies will be used to determine compatibility and rejection risk. Antibody reduction therapies (PLEX. Rituximab, proteasome inhibition) may be used to decrease the antibody burden and facilitate transplant. After transplant, monitoring for generation of new SLA antibodies or the rise of pre-formed SLA antibodies will determine recipient management and intervention to prevent xenograft rejection.

### REFERENCES

1. Sykes M. Sachs DH. Transplanting organs from pigs to humans. Sci Immunol 2019;4.
2. Hamadeh RM, Galili U, Zhou P, Griffiss JM. Anti-alpha-galactosyl immunoglobulin A (IgA), IgG, and IgM in human secretions. Clin Diagn Lab Immunol 1995;2:125-31.
3. Zappe A, Rosenlöcher J, Kohla G, Hinderlich S, Parr MK. Purification and Characterization of Antibodies Directed against the α-Gal Epitope. BioChem 2021;1:81-97.
4. Sandrin MS, Mckenzie IF. Gal alpha (1,3)Gal, the major xenoantigen(s) recognised in pigs by human natural antibodies. Immunol Rev 1994;141:169-90.
5. Dai Y, Vaught TD, Boone J, et al. Targeted disruption of the alphα1,3-galactosyltransferase gene in cloned pigs. Nat Biotechnol 2002;20:251-5.
6. Yamada K, Yazawa K. Shimizu A, et al. Marked prolongation of porcine renal xenograft survival in baboons through the use of alphα1,3-galactosyltransferase gene-knockout donors and the cotransplantation of vascularized thymic tissue. Nat Med 2005;11:32-4.
7. Kuwaki K. Tseng YL, Dor FJ, et al. Heart transplantation in baboons using alpha1,3-galactosyltransferase gene-knockout pigs as donors: initial experience. Nat Med 2005;11:29-31.
8. Montgomery RA. Stern JM, Lonze BE, et al. Results of Two Cases of Pig-to-Human Kidney Xenotransplantation. N Engl J Med 2022;386:1889-98.
9. Chen G. Qian H, Starzl T, et al. Acute rejection is associated with antibodies to non-Gal antigens in baboons using Gal-knockout pig kidneys. Nat Med 2005;11:1295-8.
10. Lu T. Yang B, Wang R, Qin C. Xenotransplantation: Current Status in Preclinical Research. Front Immunol 2019;10:3060.
11. Cooper DKC. The Long and Winding Road to Clinical Xenotransplantation—a Personal Journey. Eur Surg Res 2022.
12. Porrett PM. Orandi BJ, Kumar V, et al. First clinical-grade porcine kidney xenotransplant using a human decedent model. Am J Transplant 2022;22:1037-53.
13. Griffith BP, Goerlich CE, Singh AK, et al. Genetically Modified Porcine-to-Human Cardiac Xenotransplantation. N Engl J Med 2022.
14. Legris T, Picard C, Todorova D, et al. Antibody-Dependent NK Cell Activation Is Associated with Late Kidney Allograft Dysfunction and the Complement-Independent Alloreactive Potential of Donor-Specific Antibodies. Front Immunol 2016;7:288.
15. Mangiola M TV, Stern J. Lewis ZStewart, Lonze B, Ali N, Montgomery R. Histocompatibility Findings in the First Xenotransplants from a Pig to Deceased Human Recipient. Am J Transplant 2022;22.
16. Diaz Varela I, Sanchez Mozo P, Centeno Cortes A, Alonso Blanco C. Valdes Canedo F. Cross-reactivity between swine leukocyte antigen and human anti-HLA-specific antibodies in sensitized patients awaiting renal transplantation. J Am Soc Nephrol 2003;14:2677-83.
17. Reeves HM, Winters JL. The mechanisms of action of plasma exchange. Br J Haematol 2014;164:342-51.
18. Diaz I. Rules of thumb to obtain, isolate, and preserve porcine peripheral blood mononuclear cells. Vet Immunol Immunopathol 2022;251:110461.
19. Hatinen OA, Lahteenvuo JE, Korpela HJ, Pajula JJ, Yla-Herttuala S. Isolation of fresh endothelial cells from porcine heart for cardiovascular studies: a new fast protocol suitable for genomic, transcriptomic and cell biology studies. BMC Mol Cell Biol 2019;20:32.
20. Kaeffer B. Immortal porcine lymphoblastoid cell lines: interest for veterinary and medical research. Vet Res 1994;25:425-41.
21. Carrillo A. Chamorro S, Rodriguez-Gago M, et al. Isolation and characterization of immortalized porcine aortic endothelial cell lines. Vet Immunol Immunopathol 2002;89:91-8.
22. Allan D, Crumpton MJ. Preparation and characterization of the plasma membrane of pig lymphocytes. Biochem J 1970;120:133-43.
23. Ohkubo A, Okado T, Kurashima N, et al. Removal Characteristics of Immunoglobulin G Subclasses by Conventional Plasma Exchange and Selective Plasma Exchange. Ther Apher Dial 2015;19:361-6.
24. Dasha P, Piras AM, Dash M. Cell membrane coated nanocarriers—an efficient biomimetic platform for targeted therapy. Journal of Controlled Release 327 (2020) 546-570.
25. Narain A, Asawa S, Chhabria V, Patil-Sen Y. Cell membrane coated nanoparticles: next-generation therapeutics. Nanomedicine (Lond.) (2017) 12(21), 2677-2692.
26. Fang DH, Kroll AV. Gao W, Zhang L. Cell Membrane Coating Nanotechnology. Adv Mater. 2018 June;30(23): el 706759. doi: 10.1002/adma.201706759.
27. Ladowski JM, Hara H, Cooper DKC. The Role of SLAs in Xenotransplantation. Transplantation 2021;105(2):300-307. DOI: 10.1097/TP.0000000000003303.
28. Haji-Ghassemi O, Blackler RJ, Martin Young N, Evans SV. Antibody recognition of carbohydrate epitopesdagger. Glycobiology 2015;25(9):920-52. DOI: 10.1093/glycob/cwv037.
29. Tobian AA, Shirey RS, King KE. ABO antibody titer monitoring for incompatible renal transplantation. Transfusion 2011;51(3):454-7. DOI: 10.1111/j. 1537-2995.2011.03049.x.
30. Dehoux JP. Gianello P. Accommodation and antibodies. Transpl Immunol 2009;21(2):106-10. DOI: 10.1016/j.trim.2008.10.002.
31. Valenzuela NM, Hickey MJ, Reed EF. Antibody Subclass Repertoire and Graft Outcome Following Solid Organ Transplantation. Front Immunol 2016;7:433. DOI: 10.3389/fimmu.2016.00433.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

What is claimed is:

1. A method of predicting the risk of a subject developing a xenograft rejection response, wherein the subject is considered for a xenograft transplantation from a porcine donor, said method comprising:
a) contacting a sample obtained from the subject with a test solid or semisolid support comprising immobilized thereon cell membranes isolated from one or more porcine cells, wherein the porcine cells are isolated from the porcine donor or another animal within the same breeding family,
b) determining a titer of IgG and IgM antibodies in the sample obtained from the subject, wherein the titer is determined as the dilution at which the amount of IgG and IgM antibodies bound to the test solid or semisolid support is not more than that of a negative control sample, and
c) determining the risk of the subject developing the xenograft rejection response based on the titer determined in step (b).

2. The method of claim 1, wherein step (c) comprises determining:
(1) the subject is at a low risk of developing the xenograft rejection response when the sample has a titer of less than or equal to 1:8;
(2) the subject is at a moderate risk of developing the xenograft rejection response when the sample has a titer of higher than 1:8 but less than 1:32; or
(3) the subject is at a high risk of developing the xenograft rejection response when the sample has a titer of higher than or equal to 1:32.

3. A method of predicting the risk of a subject developing a xenograft rejection response, wherein the subject is considered for a xenograft transplantation from a porcine donor, said method comprising:
a) contacting a sample obtained from the subject with a test solid or semisolid support comprising immobilized thereon one or more Swine Leukocyte Antigens (SLA),
b) determining a titer of IgG and IgM antibodies in the sample obtained from the subject, wherein the titer is determined as the dilution at which the amount of IgG and IgM antibodies bound to the test solid or semisolid support is not more than that of a negative control sample, and
c) determining the risk of the subject developing the xenograft rejection response based on the titer determined in step (b).

4. The method of claim 3, wherein step (c) comprises determining:
(1) the subject is at a low risk of developing the xenograft rejection response when the sample has a titer of less than or equal to 1:16;
(2) the subject is at a moderate risk of developing the xenograft rejection response when the sample has a titer of higher than or equal to 1:16 but less than 1:32; or
(3) the subject is at a high risk of developing the xenograft rejection response when the sample has a titer of higher than or equal to 1:32.

5. The method of any one of claims 1-4, wherein the method further comprises proceeding with the xenograft transplantation from the porcine donor, when the subject is determined to be at a low risk of developing the xenograft rejection response.

6. The method of any one of claims 1-5, wherein the method further comprises performing a treatment to reduce the amount of anti-porcine IgG and IgM antibodies in the blood of the subject prior to performing the xenograft transplantation from the porcine donor, when the subject is determined to be at a moderate risk of developing the xenograft rejection response.

7. The method of any one of claims 1-6, wherein the method further comprises not proceeding with the xenograft transplantation from the porcine donor or performing a treatment to reduce the amount of anti-porcine IgG and IgM antibodies in the blood of the subject prior to performing the xenograft transplantation from the porcine donor, when the subject is determined to be at a high risk of developing the xenograft rejection response.

8. A method of predicting the risk of a subject developing a xenograft rejection response, wherein the subject is considered for a xenograft transplantation from a porcine donor, said method comprising:
a) contacting a sample obtained from the subject with a test solid or semisolid support comprising immobilized thereon one or more of galactose-α1,3-galactose (αGAL), N-glycolylneuraminic acid (Neu5Gc), and/or the SDa blood group (SDa),
b) determining a titer of IgG and IgM antibodies in the sample obtained from the subject, wherein the titer is determined as the dilution at which the amount of IgG and IgM antibodies bound to the test solid or semisolid support is not more than that of a negative control sample, and
c) determining the risk of the subject developing the xenograft rejection response based on the titer determined in step (b).

9. The method of claim 8, wherein the test solid or semisolid support comprises immobilized thereon Neu5Gc and/or SDa.

10. The method of claim 8 or 9, wherein step (c) comprises determining:
(1) the subject is at a low risk of developing the xenograft rejection response when the sample has a titer of less than or equal to 1:16;
(2) the subject is at a moderate risk of developing the xenograft rejection response when the sample has a titer of higher than or equal to 1:16 but less than 1:32; or
(3) the subject is at a high risk of developing the xenograft rejection response when the sample has a titer of higher than or equal to 1:32.

11. The method of any one of claims 8-10, wherein the method further comprises proceeding with the xenograft transplantation from the porcine donor, when the subject is determined to be at a low or moderate risk of developing the xenograft rejection response.

12. The method of any one of claims 8-11, wherein the method further comprises performing a treatment to reduce the amount of anti-porcine IgG and IgM antibodies in the blood of the subject prior to performing the xenograft transplantation from the porcine donor, when the subject is determined to be at a high risk of developing the xenograft rejection response.

13. The method of any one of claims 1-12, wherein the method further comprises contacting the sample obtained from the subject with
a negative control support comprising the same solid or semisolid support as in the test solid or semisolid support but without immobilized porcine cell membranes, and/or
a positive control support comprising the same solid or semisolid support as in the test solid or semisolid support with one or more immobilized porcine antigens known to cause a xenograft rejection response or react with IgM and/or IgG antibodies.

14. The method of claim 13, wherein the positive control support comprises one or more porcine antigens selected from galactose-α1,3-galactose (αGAL), N-glycolylneuraminic acid (Neu5Gc) and the SDa blood group (SDa).

15. The method of claim 14, wherein the positive control support comprises porcine antigen galactose-α1,3-galactose (αGAL).

16. The method of any one of claims 1-15, wherein the negative control sample does not have anti-porcine IgG and IgM antibodies.

17. The method of any one of claims 1-16, wherein the negative control sample does not have anti-human leukocyte antigen (HLA) antibodies.

18. The method of any one of claims 1-17, wherein the dilution is prepared by diluting the sample with a solvent.

19. The method of claim 18, wherein the solvent is a saline solution or distilled water.

20. The method of any one of claims 6, 7 and 12, wherein the treatment comprises one or more cycles of a plasma exchange.

21. The method of claim 20, wherein the method further comprises repeating steps (a)-(c) on the subject sample after said one or more cycles of the plasma exchange to determine if additional cycles of plasma exchange are needed prior to the xenograft transplantation.

22. The method of any one of claims 6, 7, and 12, wherein the treatment comprises removing plasma cells and/or B cells from the blood of the subject.

23. A method of monitoring the risk of a subject developing a xenograft rejection response, wherein the subject has received a xenograft transplantation from a porcine donor, said method comprising:
a) contacting a sample obtained from the subject shortly before the transplantation and two or more samples obtained from the subject at different time points after the transplantation with i) a test solid or semisolid support having immobilized thereon cell membranes isolated from one or more porcine cells, wherein the porcine cells are isolated from the porcine donor or another animal within the same breeding family, or ii) a test solid or semisolid support having immobilized thereon one or more Swine Leukocyte Antigens (SLA), and/or iii) a test solid or semisolid support having immobilized thereon one or more of galactose-α1,3-galactose (αGAL), N-glycolylneuraminic acid (Neu5Gc), and/or the SDa blood group (SDa);
b) determining the amount of IgG and IgM antibodies bound to the test solid or semisolid support(s) for each of the samples used in step (a);
c) comparing the antibody amounts determined in step (b), and
d) determining that
i) the subject has a decreased risk of developing a xenograft rejection response when the amount of IgG and IgM antibodies in the sample obtained at a later time point is lower than the amount of IgG and IgM antibodies in the sample obtained at an earlier time point and the sample obtained shortly before the transplantation;
ii) the subject has an unchanged risk of developing a xenograft rejection response when the amount of IgG and IgM antibodies in the sample obtained at a later time point is the same as the amount of IgG and IgM antibodies in the sample obtained at an earlier time point and the sample obtained shortly before the transplantation, or
iii) the subject has an increased risk of developing the xenograft rejection response when the amount of IgG and IgM antibodies in the sample obtained at a later time point is higher than the amount of IgG and IgM antibodies in the sample obtained at an earlier time point and the sample obtained shortly before the transplantation.

24. The method of claim 23, wherein the method comprises diluting the subject sample in a solvent from 1:2 to 1:32.

25. The method of claim 24, wherein the solvent is a saline solution or distilled water.

26. The method of any one of claims 23-25, wherein the method further comprises performing a treatment to reduce the amount of anti-porcine IgG and IgM antibodies in the blood of the subject, when the subject is determined to have an increased risk of developing the xenograft rejection response.

27. The method of claim 26, wherein the treatment comprises one or more cycles of a plasma exchange.

28. The method of claim 27, wherein the method further comprises repeating steps (a)-(d) on the subject sample after said one or more cycles of the plasma exchange to determine if additional cycles of plasma exchange are needed.

29. The method of claim 26, wherein the treatment comprises removing plasma cells and/or B cells from the blood of the subject.

30. The method of any one of claims 1-29, wherein the xenograft rejection response is an acute xenograft rejection response.

31. The method of any one of claims 1-29, wherein the xenograft rejection response is a delayed xenograft rejection response.

32. A method of selecting a porcine donor for xenograft transplantation for a subject in need thereof, said method comprising:
a) contacting a sample obtained from the subject with a plurality of differentially labeled solid or semisolid supports, each support having immobilized thereon cell membranes isolated from one or more cells of a single porcine donor from a plurality of candidate porcine donors or another animal within the same breeding family;
b) determining the amount of IgG and IgM antibodies bound to each of the differentially labeled solid or semisolid supports.
c) comparing the amounts of IgG and IgM antibodies determined in step (b) between different solid or semisolid supports, and
d) identifying a porcine donor for xenograft transplantation as the donor associated with the solid or semisolid support resulting in the lowest amount of IgG and IgM antibodies among the compared differentially labeled solid or semisolid supports.

33. The method of claim 32, wherein the method further comprises proceeding with the xenograft transplantation using the identified porcine donor.

34. The method of any one of claims 1-33, wherein the xenograft transplantation is a transplantation of a whole organ or a tissue or cellular graft.

35. The method of any one of claims 1-34, wherein the amount of IgG and IgM antibodies is determined by contacting the solid or semisolid support, after exposure to the sample, with one or more labeled detection probes which recognize the IgG and/or IgM antibodies produced by the subject, and measuring a signal produced by the label.

36. The method of claim 35, wherein the detection probe is an antibody which recognizes the IgG and/or IgM antibodies produced by the subject.

37. The method of claim 35 or claim 36, wherein the label is a fluorescent dye or mediates a chemical reaction.

38. The method of claim 37, wherein the label mediates an enzymatic reaction.

39. A method of identifying a new porcine antigen(s) recognized by anti-porcine antibodies in a subject, which antibodies mediate a xenograft rejection response in said subject to a xenograft transplantation from a porcine donor, said method comprising:
a) contacting a sample obtained from the subject with a solid or semisolid support having immobilized thereon cell membranes isolated from one or more porcine cells, wherein the porcine cells are isolated from the porcine donor or another animal within the same breeding family;
b) isolating IgG and/or IgM antibodies bound to the solid or semisolid support, and
c) determining target antigen(s) for the IgG and/or IgM antibodies isolated in step (b).

40. A method for creating a porcine xenograft donor with a minimized likelihood of inducing a xenograft rejection response, wherein the porcine donor is genetically modified to delete or inhibit the expression of the gene(s) encoding the porcine antigen(s) identified by the method of claim 39.

41. The method of any one of claims 1-40, wherein the sample is selected from serum, plasma and an immunoglobulin fraction of the blood comprising IgG and IgM immunoglobulins.

42. The method of any one of claims 1-2, 5-7, and 13-41, wherein the animal within the same breeding family is a parent of the porcine donor.

43. The method of any one of claims 1-2, 5-7, and 13-42, wherein the porcine donor is a porcine with α1,3-galactosiltransferase knock out (GTKO), a porcine with αGAL/SDa/Neu5Gc triple-knockout of α1,3-galactosiltransferase, CMP-N-acetylneuraminic acid hydroxylase and beta-1,4-N-acetyl-galactosaminyltransferase 2 (TKO), or a porcine with 10 genes modifications (10GE).

44. The method of any one of claims 1-2, 5-7, and 13-43, wherein the one or more porcine cells are one or more lymphocytes, endothelial cells, or epithelial cells, or a combination thereof.

45. The method of claim 44, wherein the lymphocytes are T cells and B cells.

46. The method of claim 44, wherein the endothelial cells are aortic endothelial cells.

47. The method of any one of claims 1-46, wherein the solid support is a bead, a particle, a microsphere, a microparticle, a plate, a microplate, a microtiter plate, a slide, a dish, a petri dish, a cup, a strand, a chip, a microchip, a strip, a membrane, a microarray, or a test tube.

48. The method of claim 47, wherein the solid support is a bead.

49. The method of claim 48, wherein the bead is a magnetic bead, plastic bead, microbead, polymer bead, or solid core bead.

50. The method of any one of claims 1-46, wherein the solid or semisolid support is a nanoparticle.

51. The method of any one of claims 1-46, wherein the semisolid support is a micelle.

52. The method of any one of claims 1-51, wherein the method is conducted in a high throughput format.

53. The method of claim 52, wherein the method is conducted in a multi-well plate.

54. The method of any one of claims 1-53, wherein the subject is human.

55. A solid or semisolid support comprising immobilized thereon i) cell membranes isolated from one or more porcine cells, or ii) one or more Swine Leukocyte Antigens (SLA), or iii) one or more galactose-α1,3-galactose (αGAL), N-glycolylneuraminic acid (Neu5Gc), and/or the SDa blood group (SDa).

56. The solid or semisolid support of claim 55, wherein the one or more porcine cells are one or more lymphocytes, endothelial cells, or epithelial cells, or a combination thereof.

57. The solid or semisolid support of claim 56, wherein the lymphocytes are T cells and B cells.

58. The solid or semisolid support of claim 56, wherein the endothelial cells are aortic endothelial cells.

59. The solid or semisolid support of any one of claims 55-58, wherein the porcine cells are isolated from a porcine with α1,3-galactosiltransferase knock out (GTKO), a porcine with αGAL/SDa/Neu5Gc triple-knockout of α1,3-galactosiltransferase, CMP-N-acetylneuraminic acid hydroxylase and beta-1,4-N-acetyl-galactosaminyltransferase 2 (TKO), or a porcine with 10 genes modifications (10GE).

60. The solid or semisolid support of any one of claims 55-59, wherein the solid support is a bead, a particle, a microparticle, a plate, a microplate, a microtiter plate, a slide, a dish, a petri dish, a cup, a strand, a chip, a microchip, a strip, a membrane, a microarray, or a test tube.

61. The solid or semisolid support of claim 60, wherein the solid support is a bead.

62. The solid or semisolid support of claim 61, wherein the bead is a magnetic bead, microbead, polymer bead, or solid core bead.

63. The solid or semisolid support of any one of claims 55-59, wherein the solid or semisolid support is a nanoparticle.

64. The solid or semisolid support of any one of claims 55-59, wherein the semisolid support is a micelle.

65. The solid or semisolid support of any one of claims 55-64, wherein the semisolid or solid support is comprised within a multi-well plate.

66. A kit comprising
(i) the solid or semisolid support of any one of claims 55-65;
(ii) optionally, a positive control support comprising the same solid or semisolid support as in the test solid or semisolid support with one or more immobilized porcine antigens known to cause a xenograft rejection response or react with IgM and/or IgG antibodies;
(iii) optionally, a negative control support comprising the same solid or semisolid support as in the test solid or semisolid support but without immobilized porcine cell membranes;
(iv) optionally, a labeled detection probe which recognizes IgG and/or IgM antibodies; and
(v) optionally, packaging and/or instructions for using the same.

67. The kit of claim 66, wherein the one or more porcine antigens are selected from galactose-α1,3-galactose (αGAL), N-glycolyIncuraminic acid (Neu5Gc) and the SDa blood group (SDa).

68. The kit of claim 66 or claim 67, wherein the detection probe is an antibody.

69. The kit of any one of claims 66-68, wherein the label is a fluorescent dye or mediates a chemical reaction.

70. The kit of claim 69, wherein the label mediates an enzymatic reaction.
